(19) United States
(12) Patent Application Publication
(10) Pub. No.: US 2024/0387021 A1
(43) Pub. Date: Nov. 21, 2024

(54) SYSTEMS AND METHODS FOR AUTOMATING DELIVERY OF MENTAL HEALTH THERAPY

(71) Applicant: OPTT Health, Inc., Boston, MA (US)

(72) Inventors: Amirhossein SHIRAZI, Quebec (CA); Mohsen OMRANI, Ontario (CA)

(73) Assignee: OPTT Health, Inc., Boston, MA (US)

(21) Appl. No.: 18/692,211

(22) PCT Filed: Sep. 14, 2022
(86) PCT No.: PCT/US2022/043514

§ 371 Date: Mar. 14, 2024

Related U.S. Application Data
(60) Provisional application No. 63/244,436, filed on Sep. 15, 2021.

(51) Int. Cl. G16H 20/70; G16H 10/20; G16H 10/60; G16H 15/00; G16H 20/10; G16H 50/20; G16H 50/30

(52) U.S. Cl. CPC G16H 20/70; G16H 10/20; G16H 10/60; G16H 15/00; G16H 20/10; G16H 50/20; G16H 50/30

(57) ABSTRACT

Systems and methods for evaluating individual's mental health is provided. The mental health status is provided through an analysis of textual data to identify elemental factors relevant to mental health. The analysis of patients' textual data is performed based on the answers to one or more homework that contain symptom-related (e.g., depression or anxiety) contents.

### CROSS-REFERENCE TO RELATED APPLICATIONS

The present application claims the benefit of U.S. Provisional Application No. 63/244,436, filed on Sep. 15, 2021, the contents of which are incorporated herein by reference as if set forth in full.

### FIELD OF INVENTION

This invention is related to systems and methods for evaluating individual's mental status through an analysis of textual data to identify elemental factors relevant to mental health.

### BACKGROUND

Speech is the primary medium of diagnosis and intervention in today's psychiatry. It works better from the diagnostic perspective than any other diagnostic equipment (e.g., imaging, genetic or molecular tests). On the other hand, from an interventional standpoint, psychotherapy and talk therapy is the gold standard and first line of treatment for many mental health problems over medical intervention. Therefore, analyzing speech and quantifying mental health problems using speech can be a potent diagnostic and prognostic tool. A clinician tries to pinpoint the source of symptoms through a clinical interview, verify ill-formed patterns (schemata), and intervene to change them. This structured analysis of an individual's speech allows impartial consideration of speech content, takes into account its form, and is very different from our normal enacts in response to a conversation. While clinicians receive multi-year training to conduct such analysis, this process remains in the subjective domain and unscalable.

Despite significant breakthroughs and advanced new techniques in diagnosing and treating diseases in different medical fields, mental health problems are still diagnosed and treated with outdated methods and medications. A major flaw is the lack of rigorous and objective characterization of mental health problems rather than anecdotal symptomatology and disorder labeling. This somewhat arbitrary pooling of different symptoms under the same disorder name, without enough characterization, results in medications that only work for a fraction of the patients and essentially >50% waste of resources for the rest. There is a need to exploit new capacities provided by the digital delivery of mental healthcare and provide a system or method for implementing a robust set of evaluation techniques that allow objective characterization of patients' mental status.

### SUMMARY

A method of providing mental health care is provided. The method includes obtaining textual data from a patient; analyzing the textual data via an algorithm to generate predictive contents, wherein the predictive contents comprise a prediction of a diagnostic condition, a prediction of a symptomatic score, and a prediction of a compliance level of the patient; predicting a mental status of the patient in accordance with the predictive contents; and providing a modality of mental health treatment based on the predicted mental status of the patient.

A system for providing mental health care is provided. The system includes an algorithm to analyze textual data to generate predictive contents, wherein the predictive contents comprise a prediction of a diagnostic condition, a prediction of a symptomatic score, and a prediction of a compliance level of the patient. The system is configured to predict a mental status of the patient in accordance with the predictive contents and to provide a modality of mental health treatment based on the predicted mental status of the patient.

A method of providing mental health therapy is provided. The method includes obtaining textual data from a patient enrolled in therapy; analyzing the textual data via an algorithm to generate predictive contents, wherein the predictive contents comprise a prediction of a diagnostic condition, a prediction of a symptomatic score, and a prediction of a compliance level of the patient in the therapy; and predicting an outcome of the therapy in accordance with the predictive contents.

A system for providing mental health therapy is provided. The system includes an algorithm to analyze textual data from a patient enrolled in therapy to generate predictive contents, wherein the predictive contents comprise a prediction of a diagnostic condition, a prediction of a symptomatic score, and a prediction of a compliance level of the patient in the therapy. The system is configured to predict an outcome of the therapy in accordance with the predictive contents.

A method of providing psychiatric feedback is provided. The method includes obtaining textual data from a patient; analyzing the textual data via an algorithm to select one or more relevant phrases or sentences, wherein the one or more relevant phrases or sentences are selected by adjusting a threshold value for one or more of a physical condition, a mental condition, or an emotional condition; summarizing the one or more relevant phrases or sentences to produce a summary of a patient condition; and generating a psychiatric feedback based on the patient condition summary.

A system for providing psychiatric feedback is provided. The system includes an algorithm to analyze textual data from a patient to select one or more relevant phrases or sentences, wherein the one or more relevant phrases or sentences are selected by adjusting a threshold value for one or more of a physical condition, a mental condition, or an emotional condition. The system is configured to summarize the one or more relevant phrases or sentences to produce a summary of a patient condition and to generate a psychiatric feedback based on the patient condition summary.

### BRIEF DESCRIPTION OF THE DRAWINGS

This accompanying drawings are not intended to be drawn to scale. Like reference numbers and designations in the various drawings indicate like elements. For purposes of clarity, not every component may be labeled in every drawing. In the drawings:

FIG. 1 illustrates a system for providing a treatment suggestion through an analysis of textual data, according to various embodiments.

FIG. 2 illustrates a system for predicting a therapy outcome through an analysis of textual data, according to various embodiments.

FIG. 3 illustrates a system for predicting psychiatric feedback through an analysis of textual data, according to various embodiments.

FIGS. 4A and 4B illustrates respective flow charts illustrating example methods for evaluating mental status using textual data to identify elemental factors relevant to mental health, according to various embodiments.

FIG. 4C illustrates an example output, according to various embodiments.

FIGS. 5A and 5B illustrates how sentences are parsed, according to various embodiments.

FIGS. 6A, 6B, and 6C illustrate relevancy of each sentence in an essay with depression and positive thoughts, according to various embodiments.

FIGS. 7A-7F illustrate various plots generated by an algorithm, according to various embodiments.

FIG. 8 illustrates a method for providing a treatment suggestion through an analysis of textual data, according to various embodiments.

FIG. 9 illustrates a method for predicting a therapy outcome through an analysis of textual data, according to various embodiments.

FIG. 10 illustrates a method for predicting psychiatric feedback through an analysis of textual data, according to various embodiments.

FIG. 11 is a block diagram of a computing system configured to perform an analysis of textual data, in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

In order to make mental health evaluation more objective and also scalable, natural language processing (NLP) algorithms are used to extract clinically relevant elemental features of a patient's textual data. The algorithms can be implemented to assist clinicians in preparing personalized feedback to their patients, suggest the most appropriate resources needed for a patients' therapy, and predict an intervention's outcome. Through this process, the individual's mental status can be remotely monitored in a continuous and long-term fashion and predict and prevent future mental health crises.

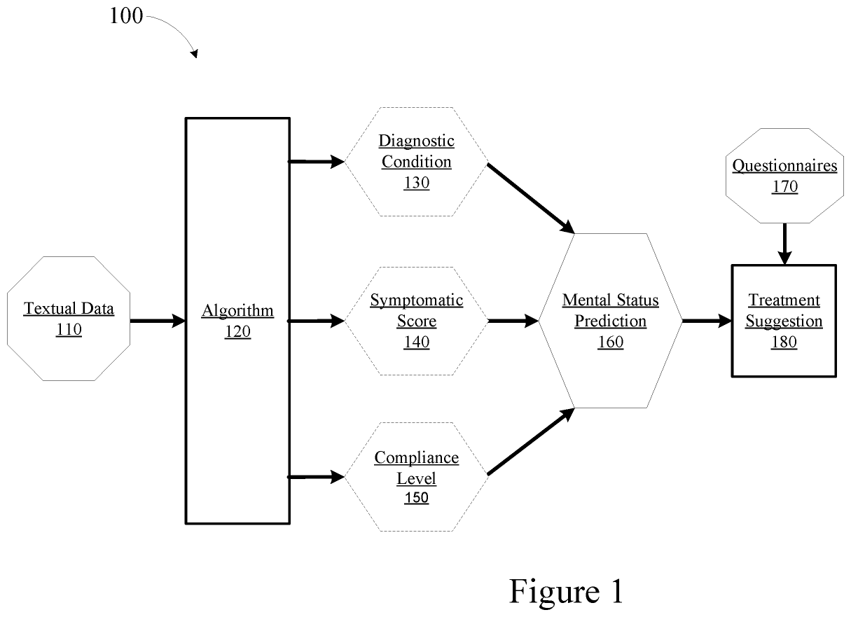

FIG. 1 illustrates a system 100 for providing a treatment suggestion through an analysis of textual data, according to various embodiments. As illustrated in FIG. 1, the system 100 is a system for providing mental health care. The system 100 includes an algorithm 120 (also referred to herein as an artificial intelligence “AI” or a “model”, etc.). When a textual data 110 from a patient (e.g., as part of a homework) is analyzed via the algorithm 120, the system 100 (via the algorithm 120) can generate one or more predictive contents. As illustrated in FIG. 1, the predictive contents can be any number of predictions, for example, a prediction of a diagnostic condition 130, a prediction of a symptomatic score 140, and/or a prediction of a compliance level 150 of the patient. In various embodiments, the system 100 (via the algorithm 120) can further predict a mental status 160 of the patient based on or in accordance with any number of the predictive contents. Using the algorithm-predicted mental status 160 of the patient, a clinician or a therapist can provide a treatment suggestion 180, e.g., modality of mental health treatment, to the patient based on the predicted mental status.

In various embodiments, when providing the modality of mental health treatment, e.g., the treatment suggestion 180, the clinician or therapist can take into consideration a score from a set of quantitative measurements 170 (also referred to herein as Questionnaires 170) designed for mental health therapy. For example, the set of quantitative measurements that can be used by the clinician or therapist can include Patient Health Questionnaire-9 (PHQ-9), which is designed for depression, General Anxiety Disorder-7 (GAD-7), which is designed for generalized anxiety, or Quality of Life Enjoyment and Satisfaction Questionnaire-18 (Q-LES), which is designed for quality of life. In various embodiments, the modality of the mental health treatment, or simply, treatment suggestion can be determined based on the predicted mental status of the patient and the score from one of PHQ-7, GAD-7, or Q-LES. Additional details are provided further below.

In various embodiments, the prediction of the diagnostic condition 130 can include a prediction of a physical symptom, an emotional symptom or a physical condition. In various embodiments, the physical symptom includes panic attack or anxiety. In various embodiments, the emotional symptom includes a feeling of isolation or a feeling of social phobia. In various embodiments, the physical condition includes a difficulty of sleeping or insomnia.

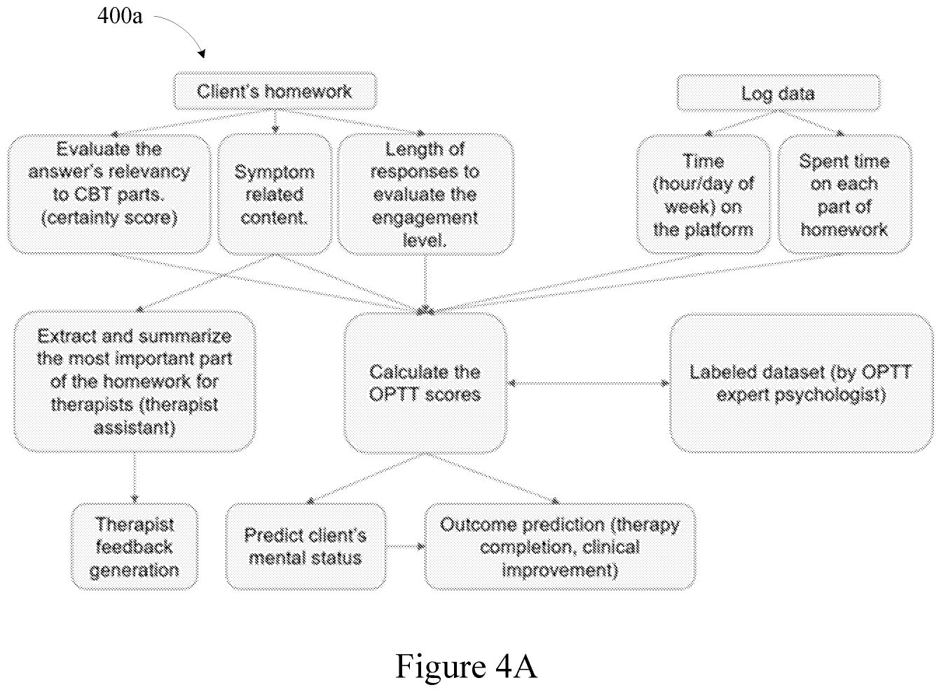

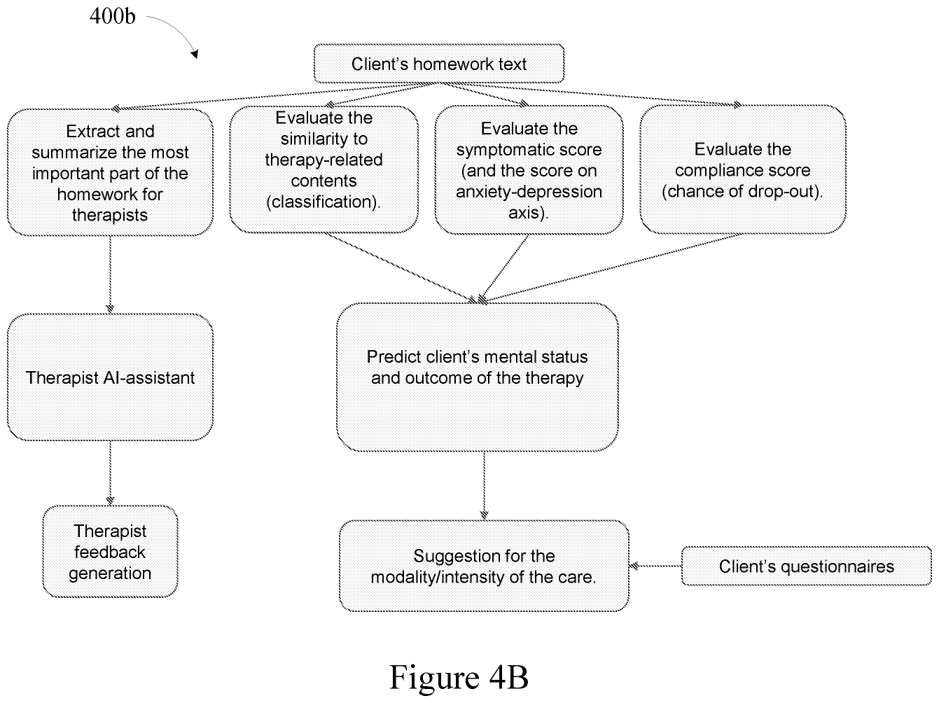

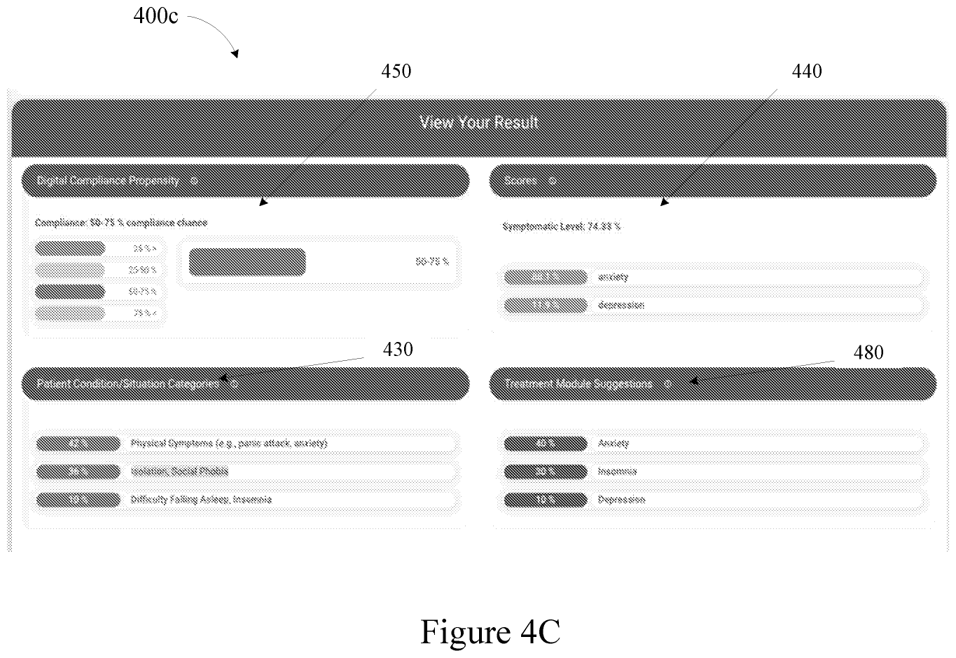

In various embodiments, the prediction of the symptomatic score 140 can include a percentage prediction for a predicted diagnostic condition. In various embodiments, the compliance level 150 of the patient can be a propensity or a likelihood that the patient stays within a therapy program. Further details related to the prediction of the diagnostic condition 130, the prediction of the symptomatic score 140, the compliance level 150 or propensity of the patient are illustrated in an example output 400c of FIG. 4C, where chart 430 shows example predictions of the diagnostic condition 130, chart 440 shows example predictions of the symptomatic score 140, and chart 450 shows example compliance level 150 or propensity of the patient, respectively. Moreover, FIG. 4C also shows treatment module suggestions in chart 480.

In various embodiments, the algorithm 120 is a natural language processing algorithm trained to evaluate a relationship of a textual statement in accordance with a five-part model of cognitive behavioral therapy that recognizes situation (positive or negative), behavior, thought (positive or negative), emotion (positive or negative), and physical reaction. In various embodiments, deep-learning machine learning models (natural language processing (NLP)) algorithm are used to calculate the similarity of sentences or phrases. In various embodiments, technique called Natural Language Inference (NLI) can be used for calculation.

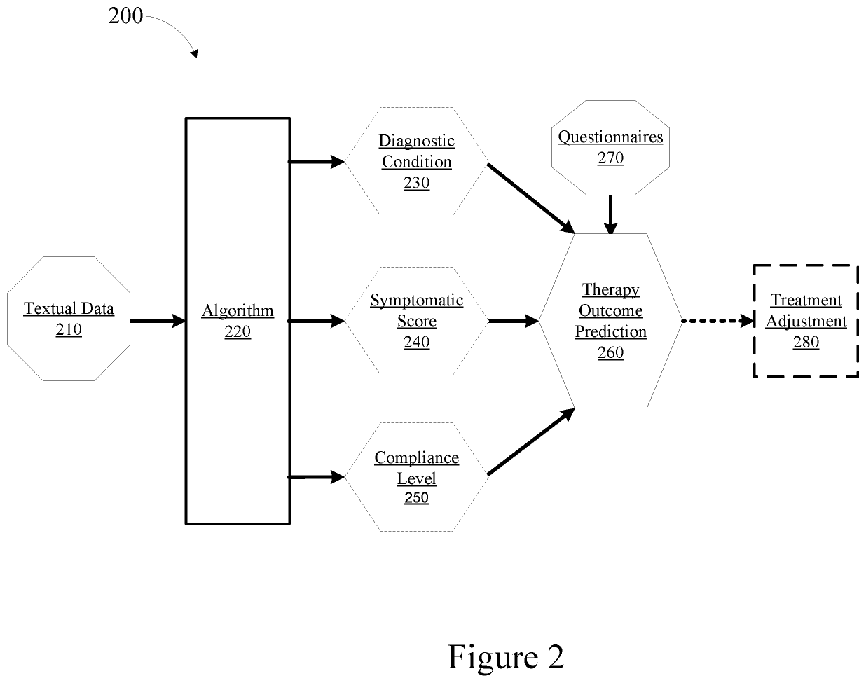

FIG. 2 illustrates a system 200 for predicting a therapy outcome through an analysis of textual data, according to various embodiments. As illustrated in FIG. 2, the system 200 is a system for providing mental health therapy. The system 200 includes an algorithm 220. When a textual data 210 from a patient is analyzed via the algorithm 220, the system 200 (via the algorithm 220) can generate one or more predictive contents. As illustrated in FIG. 2, the predictive contents can be any number of predictions, for example, a prediction of a diagnostic condition 230, a prediction of a symptomatic score 240, and/or a prediction of a compliance level 250 of the patient. In various embodiments, the system 200 (via the algorithm 220) can further predict an outcome of the therapy in accordance with the predictive contents.

In various embodiments, when predicting the outcome of the therapy, the clinician or therapist can take into consideration a score related to a set of quantitative measurements 270 (also referred to herein as Questionnaires 270) designed for mental health therapy. In various embodiments, the set of quantitative measurements comprises PHQ-9, GAD-7, or Q-LES. In various embodiments, the outcome of the therapy is predicted based on the symptomatic score and the score from one of PHQ-7, GAD-7, or Q-LES.

In various embodiments, the prediction of the diagnostic condition 230 can include a prediction of a physical symptom, an emotional symptom or a physical condition. In various embodiments, the physical symptom includes panic attack or anxiety. In various embodiments, the emotional symptom includes a feeling of isolation or a feeling of social phobia. In various embodiments, the physical condition includes a difficulty of sleeping or insomnia.

In various embodiments, the prediction of the symptomatic score 240 comprises a percentage prediction for a predicted diagnostic condition. In various embodiments, the compliance level 250 of the patient is a propensity or a likelihood that the patient stays in therapy. Further details related to the prediction of the diagnostic condition 230, the prediction of the symptomatic score 240, the compliance level 250 or propensity of the patient are illustrated in an example output 400c of FIG. 4C, where chart 430 shows example predictions of the diagnostic condition 230, chart 440 shows example predictions of the symptomatic score 240, and chart 450 shows example compliance level 250 or propensity of the patient, respectively.

In various embodiments, the algorithm 220 is a natural language processing algorithm trained to evaluate a relationship of a textual statement in accordance with a five-part model of cognitive behavioral therapy that recognizes situation (positive or negative), behavior, thought (positive or negative), emotion (positive or negative), and physical reaction. In various embodiments, deep-learning machine learning models (natural language processing (NLP)) algorithm are used to calculate the similarity of sentences or phrases. In various embodiments, technique called Natural Language Inference (NLI) can be used for calculation.

In various embodiments, the predicted outcome of the therapy includes whether the patient completes the therapy or whether a mental status of the patient would significantly change following therapy. In various embodiments, the system 200 can include adjusting a treatment 280 (i.e., treatment adjustment 280) of the patient based on the predicted outcome of the therapy. In various embodiments, the treatment adjustment 280 of the patient can include escalating an intervention level or adding medication to the therapy.

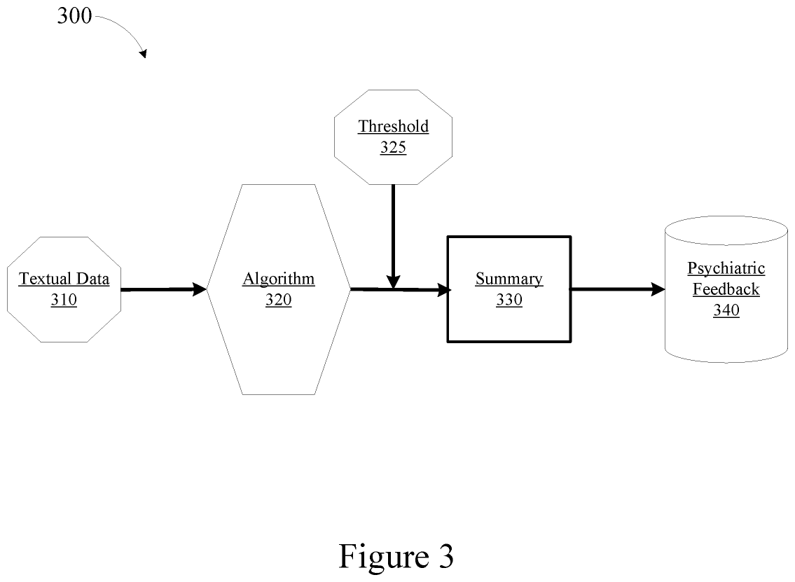

FIG. 3 illustrates a system 300 for predicting psychiatric feedback through an analysis of textual data, according to various embodiments. As illustrated in FIG. 3, the system 300 includes an algorithm 320. When a textual data 310 from a patient is analyzed via the algorithm 320, the system 300 (via the algorithm 320) can select one or more relevant phrases or sentences by adjusting a threshold 325. In various embodiments, the one or more relevant phrases or sentences are selected by adjusting the threshold 325 (e.g., adjusting threshold value via a slider bar or by inputting desired ranges or values) for one or more of a physical condition, a mental condition, or an emotional condition. In various embodiments, the system 300 can combine the selected sentences or phrases to form a summary of a patient condition (e.g., by summarizing the one or more relevant phrases or sentence). In various embodiments, the system 300 can then generate a psychiatric feedback based on the summary of the patient condition. In various embodiments, the generated psychiatric feedback can be used for providing a psychiatric therapy to the patient. In various embodiments, the system 300 may optionally include analyzing the textual data via the algorithm 320 to generate predictive contents. In various embodiments, the predictive contents may include a prediction of a diagnostic condition, such as diagnostic condition 130 or 230, and a prediction of a symptomatic score, such as symptomatic score 140 or 240. In various embodiments, the predictive contents can then be used for providing psychiatric therapy to the patient.

FIGS. 4A and 4B illustrates respective flow charts 400a and 400b illustrating example methods for evaluating mental status using textual data to identify elemental factors relevant to mental health, and FIG. 4C illustrates an example output 400c, in accordance with various embodiments of the present disclosure.

Elemental feature extraction-Patients are instructed to go through weekly online learning modules and are prompted to explain a situation that caused them great anxiety/depression and analyze it using what they learned that week. A proprietary machine-learning algorithm (Clinician-Assist algorithm) is used to analyse textual data and extract elemental features of their thinking patterns, behaviors, emotions, and physical reactions in a given situation. The algorithm also analyzes whether symptoms and sentences are in the context of anxiety or depression and flags self-harming behaviors and suicidal ideations.

Clinician feedback preparation—Using the elemental features extracted, and following session specific templates, the algorithm prepares a personalized draft used by the clinician to provide feedback to their patients. This helps clinicians to increase their efficiency in providing feedback to patients.

Patient mental status evaluation—Using elemental features extracted from patients' homework, and the scores from other standardized clinical questionnaires, the algorithm generates a mental status score that provides an objective evaluation of a patient's mental status. This measure could be used to evaluate a patient's mental status remotely and longitudinally.

Patient triage—Using the customized mental status score and historical proprietary data, the algorithm can suggest the amount and depth of care each patient needs.

Treatment outcome prediction—Using customized mental status score and our historical proprietary data, the algorithm can predict the treatment outcome (e.g., patient compliance).

Treatment customization suggestions—Using treatment outcome prediction generated by the algorithm and historical proprietary data, the algorithm can suggest modification to a patient's care plan (e.g., add medication) to improve the quality of the care (e.g., increase patient compliance).

The disclosure of the present application relates to systems and methods for analyzing patients' textual data to determine answers to clinically fundamental questions relevant in cognitive behavioral therapy, for example, but not limited to a five-part model, that includes for example, situation, behavior, thought, emotion, and physical reaction. The textual data in patient's homework may contain symptom-related contents, such as, for example, but not limited to depression or anxiety, or other contents. The systems and methods include, in various embodiments, extraction of answers from the textual data and infer the level of patients' engagement and understanding of their task.

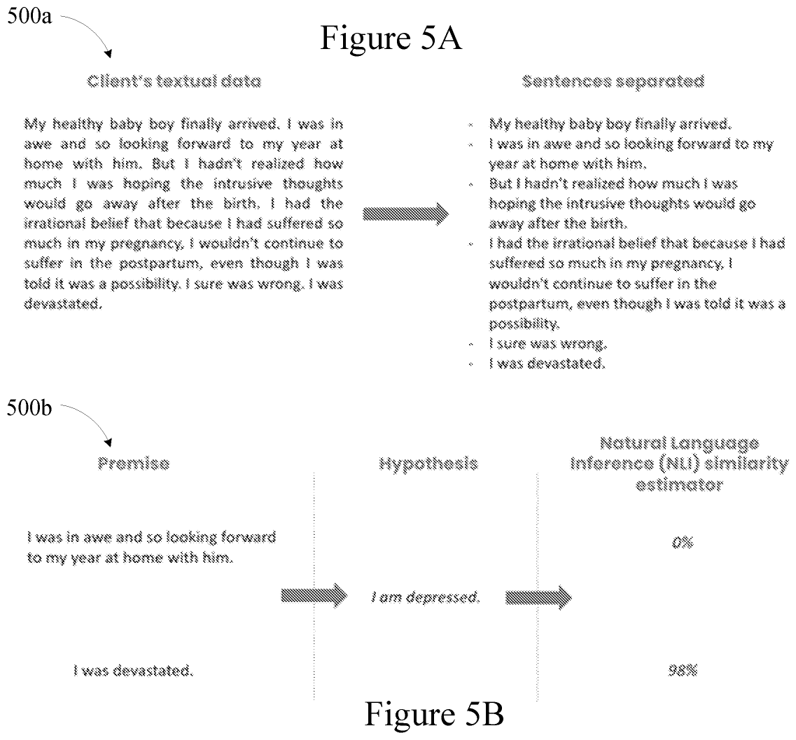

Determining symptom related content-Patient's homework can be parsed into separate sentences or sections using an off the shelf algorithm of sentence tokenization, using markers like “.”, “?”, . . . . See FIG. 5A. Deep learning machine learning models (Transformers” models) natural language processing (NLP) algorithm is then used to calculate the similarity of that sentence/section to a set of phrases. This comparison uses a technique called Natural Language Inference (NLI), through which the algorithm considers two sentences: a “premise” and a “hypothesis” and determines whether the hypothesis is true (entailment) or false (contradiction) given the premise. See FIG. 5B below. The NLI pre-trained deep learning model can return the certainty score which can be inferred to as the relevancy of the hypothesis and the premise (the score is between 0 and 1, higher value indicates higher relevancy/entailment). For instance, when comparing the premise, “I have been practicing the whole day”, with the hypothesis that I am “tired”, the certainty score is 0.93 but compared to the hypothesis that I am “happy”, the certainty score is 0.10.

While this off the shelf NLI algorithm provides the probability of similarity between two concepts, the algorithm is neither sensitive nor specific enough to be used for clinical purposes. Furthermore, a big clinical dataset to train similar deep learning Transformers for this purpose, are scarce. To address these problems, instead of retraining a deep learning model from scratch or finding a model that works for clinical data out of the box, a machine learning algorithm on top of the NLI Transformers model may be implemented.

Using this algorithm, the relevancy of each sentence to symptoms is calculated. The symptoms include, for example, Depression and Anxiety or the five-part model of cognitive behavioral therapy (CBT) like Situation (positive or negative), Behavior, Thought (positive or negative), Emotion (positive or negative), and Physical reaction.

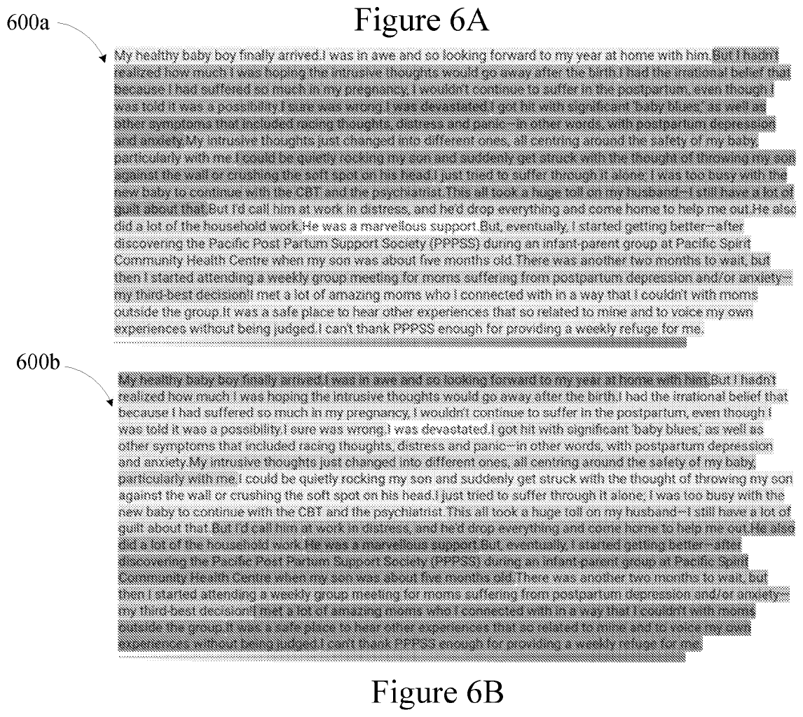

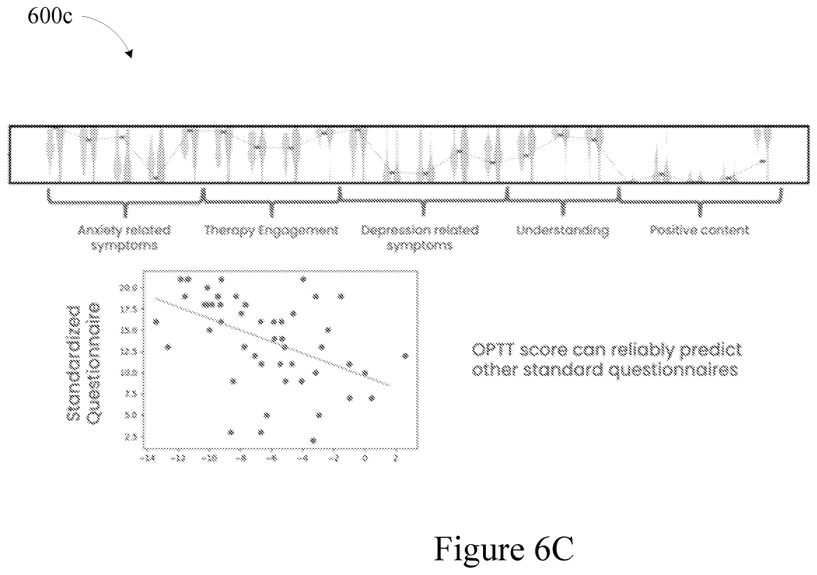

FIGS. 6A, 6B, and 6C illustrate relevancy of each sentence in an essay with depression and positive thoughts, according to various embodiments. Different shades of red reflect the magnitude of relevancy to each phrase with white representing 0% relevance and deep red representing 100%. For example, FIG. 6A shows varying levels of relevance to “depression”, whereas FIG. 6B shows varying levels of relevance to “positive thoughts”.

Note that while the algorithm is blind to the opposite meaning of these two phrases, it assigned almost opposite relevancy scores to each sentence, either it is relevant to depression or relevant to the positive thought.

Extract and summarize client's assignment—To make it easier for the clinician to read the client's assignment, the symptom-related sections of the assignment are highlighted. The concept/symptom relevance of each sentence (e.g., depression-related, anxiety-related, etc.) is calculated and highlighted if it is greater than a threshold. The clinician interface includes a rangebar to change this threshold level, for each concept/symptom. This way the clinician has the control to see the most relevant information first. Based on the proportion of sentences that reflect depressive/anxiety symptoms and the probability score each sentence receives, a score is calculated (i.e., OPTT symptomatic score)

In various embodiments, the highlighted sentences corresponding to each concept/symptom are collected and a summary is prepared using a pre-trained summarizer model (off the shelf). These summaries are fitted into predetermined spaces in a predesigned feedback template to prepare a personalized feedback for that specific client.

Determine the relevancy of answers—In a homework assignment, clients are asked to break down a psychological challenging experience into some elemental components (e.g., the situation of the experience, feelings and physical reactions clients experienced in that situation, the thought that preoccupied their mind and their behavioral responses in that situation). A proprietary algorithm is implemented to measure the relevancy level of an answer provided by the clients to a specific question, which is referred to herein as “OPTT relevancy score” (e.g., for the “Describe the situation” question, clients' answer is evaluated on how well it describes the “Situation” rather than “Physiological reaction”). This provides a measure on how much the client understood the task required from them.

Determine the client engagement—In order to evaluate clients' engagement with their therapy, a comparison is performed. Based on this comparison, an engagement score of an answer is defined as the odd ratio of probability belonging to the engaged group vs the unengaged group. In various embodiments, log data are collected and combined; the log data includes user interaction with the analysis platform, including number of logging to the platform during a week, the time of the day/day of the week of services rendered, the time they spent on each part of the homework (i.e., to read the material and to write responses), and also the length and frequency of interaction with live agents on the platform (e.g., chat or video communication). All this information is combined to generate “OPTT engagement score”. The measurements between clients are compared and labeled as engaged vs. unengaged through their therapy. As described above, an engagement score would be the odd ratio of probability belonging to the engaged group vs the unengaged group.

Mental status evaluation—A regression model is applied, in order to be able to predict a client's mental status based on the variables extracted above. Several clinical questionnaires from the clients are collected at different timepoints to have a quantitative measurement of their mental status during therapy (PHQ-9 for depression, GAD-7 for generalized anxiety, Q-LES for quality of life, etc.). A regression model is then trained using variables extracted mentioned above (e.g., prevalence of symptom related content or client engagement score) to predict their questionnaire scores. Given that evidence-based categorization for diagnosis and severity of mental health disorders are already available for these questionnaires (e.g., PHQ-9 scores between 5-9 suggest mild depression, 10-14, 15-19 and 20-27 suggest moderate, moderately severe and severe respectively), the calculated score based on extracted variables are used to diagnose and determine the severity of new client's mental health status, without asking them to complete any questionnaire. These scores are referred to herein as “OPTT mental status scores”.

Outcome prediction—All calculated variables and historic dataset can be used to predict different outcomes for each client. For instance, to determine if a client would complete the whole round of therapy or drop out in the middle, a classification model based on the variables extracted above and previous clients' outcome is trained. At this point, a prediction is made whether a patient completes the therapy and if their mental status would significantly change following therapy. This information could help clinicians adjust their approach (e.g., escalate intervention level or add medication) if the preferred outcome is not expected.

A single client's homework(s) can be analyzed to calculate different OPTT Scores. Then each individual homework is compared across all previous clients' homework with different outcomes. For example, in the above example, orange violin plots show the distribution of scores in each class among patients not showing significant clinical improvement while green violin plots show the score distribution among patients with significant clinical improvement. Based on these distributions, the probability of whether this specific client will improve or not is determined.

Multiple clinical trials are conducted to evaluate the efficacy of e-CBT for depression and anxiety, using an asynchronous textual medium. In these trials, the therapist-guided, e-CBT program for depression and anxiety consisted of 12-weeks. The results of these three trials (n=190) provide a comprehensive set of textual data, standard questionnaire scores (e.g., PHQ-9), and clinical outcomes (e.g., completion vs. dropout). Using a proprietary NLP algorithm, a comprehensive dataset (n=190) is used to calculate Symptomatic Scores for each participant.

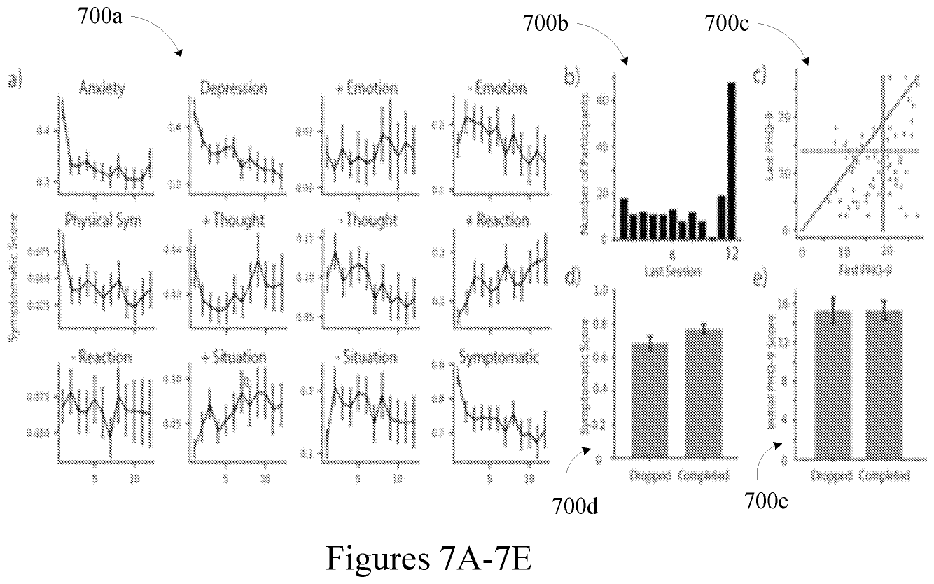

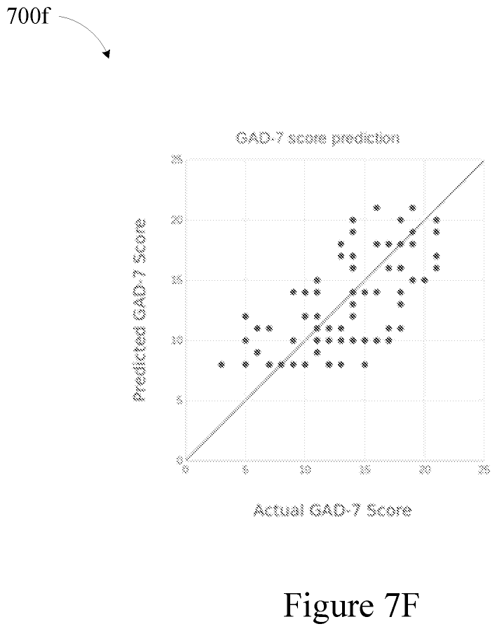

FIGS. 7A-7F illustrate various plots generated by an algorithm, such as algorithms 120, 220, or 320, according to various embodiments. FIG. 7A shows plots 700a illustrating the average Symptomatic Score (score ranging between 0-1) of these participants' homework, where they described their personal challenges, across the 12 weeks of therapy. The correlation between the participants' first homework Symptomatic Scores and their clinical outcomes (e.g., significant changes in questionnaire scores or therapy completion) are explored.

An important challenge in mental healthcare is to increase patient compliance and therapy completion. FIG. 7B shows a histogram 700b of the number of sessions completed by participants in the trials mentioned above. In this figure approximately 21% (n=41) of participants dropped out before session 4. An interesting question is whether the participants' Symptomatic Scores in the first session could predict participant compliance. A significant difference is observed as shown in plot 700d of FIG. 7D in the Symptomatic Score between the participants that dropped out before session 4 and those who completed therapy (FIG. 7D, T-score=−2.33, p=0.02, independent sample t-test). Interestingly, the initial PHQ-9 score across these two groups are not significantly different as shown in plot 700e of FIG. 7E, T-score=−0.25, p=0.79, independent sample t-test. This suggests that while a standard questionnaire, like the PHQ-9, cannot predict patient compliance, parameters extracted from participants' textual data can make such predictions, as illustrated herein.

Furthermore, FIG. 7A illustrate symptomatic score analysis of weekly homework from 190 participants through the 12 weeks of their therapy. Each panel shows a symptomatic score for a specific symptom (e.g., anxiety). Symptomatic score is lowered through the 12 weeks for negative symptoms (e.g., depression) but increased for positive ones (e.g., +reaction). Error bars demonstrate between-subject weekly score SEM. FIG. 7B shows a histogram of the number of sessions completed by participants through previous clinical trials. While 45% of participants completed the whole round of therapy, ˜21% of participants dropped out before session 4. FIG. 7C depicts a relationship between participants' initial PHQ-9 score vs. their final PHQ-9 score after completing the round of therapy. Most participants showed improvement in their clinical symptoms as evaluated by PHQ-9 (i.e., points below the unity line). On average, a full round of e-CBT lowered the PHQ-9 score by 5 points. Nevertheless, some participants fall into moderately severe depressed (i.e., PHQ-9>14, green line) at the end of the therapy. In some embodiments, the level of care is increased for those patients who's initial PHQ-9 score is higher than 19 (red line). FIG. 7D shows average symptomatic scores of session 1 homework in participants dropping out before session 4 (i.e. ‘Dropped’) and those completed. FIG. 7E shows an average initial PHQ-9 scores compared across participants who Dropped versus Completed. Error bars demonstrate between-subject SEMs. While there is a significant difference between first session symptomatic scores among participants who Dropped versus Completed, no such significance was observed in their initial PHQ-9 scores. FIG. 7F shows a plot 700f for an algorithm trained in the main task, which can predict the GAD-7 score (Pearson correlation r=0.65, r-square=40%, n=120).

Triage—A Triage module is designed to gather the relevant data (i.e., participant compliance and change in depression severity, as evaluated by the PHQ-9). As explained herein, NLP of the participants' written accounts of their challenges with depression in the Triage module will be used to calculate a Symptomatic Score. As indicated, if the PHQ-9 score <19 and the Symptomatic Score >0.75, the participant will be assigned to the e-CBT only treatment group. However, if either the PHQ-9 score >19 or the Symptomatic Score <0.75, the participants will be assigned to the e-CBT treatment with weekly phone/video calls. If both scenarios occur and the PHQ-9 score >19 and Symptomatic Score <0.75, then the participant will be assigned to the e-CBT treatment with weekly video calls and psychiatrist intervention.

To verify the algorithm's treatment allocation logic, the completion rate and the change in PHQ-9 scores are assessed in a sample of participants (n=190) who were previously enrolled in e-CBT-only treatment. A decision-making algorithm determined that the e-CBT-only program was suitable for 62 out of the 190 participants (33%). Within these 62 participants, 60% had completed the e-CBT-only program in its entirety and only 20% had a final PHQ-9 score >14. Furthermore, the algorithm indicated that e-CBT with weekly engagements would be suitable for 100 out of the 190 participants (53%). Of the 100 participants, 45% completed the whole round of e-CBT-only therapy and 31% had a final PHQ-9 score >14. Lastly, the algorithm indicated that e-CBT with video call+psychiatrist intervention was appropriate for 28 out of 190 participants (14%). Of these 28 participants, 35% completed the whole round of e-CBT-only therapy and 40% had a final PHQ-9 score >14. The logic of the algorithm's decision is therefore justified as those participants allocated to the e-CBT-only group had the highest percentage of completion and lowest percentage of final PHQ-9 scores >14 when completing e-CBT-only. Therefore, minimal therapist intensity is required for these individuals and e-CBT-only is sufficient. Conversely, participants allocated to the e-CBT with video call+psychiatrist intervention had the lowest completion rates and highest rates of final PHQ-9 scores >14 when enrolled in e-CBT-only. These findings justify the algorithm's logic that greater therapist interaction is required. It is also important to note that demographic factors like age (below or above 40 years), sex (male or female) and income (less or more than $50K) did not have any significant effects on the number of sessions completed by participants (p=0.92, 0.18 & 0.9 for age, sex, and income respectively). The demographic factors did not affect the change in PHQ-9 score (i.e., difference between the beginning and end of treatment scores) either (p=0.2, 0.46 & 0.39 for age, sex and income respectively).

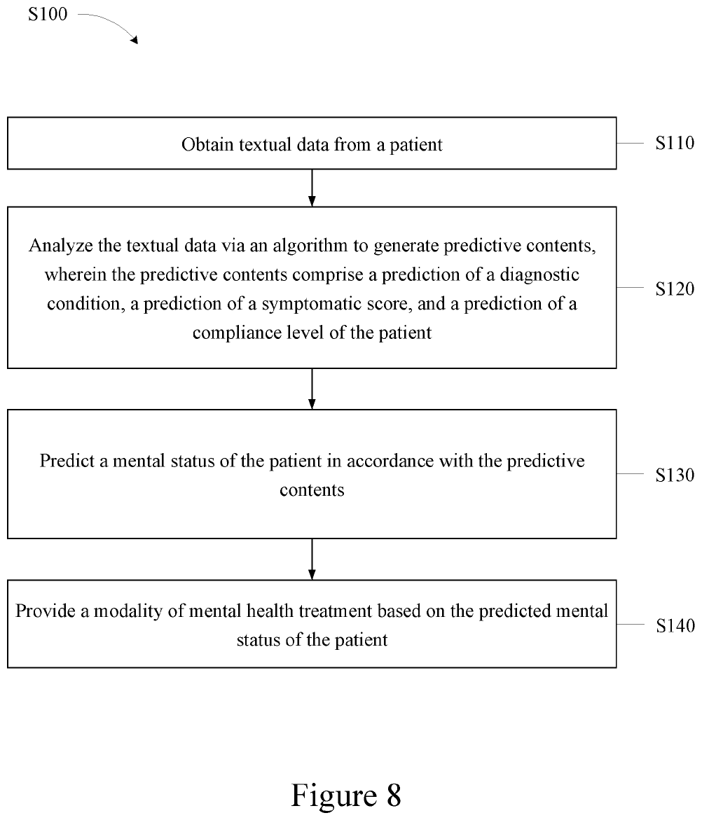

FIG. 8 illustrates a method S100 for providing a treatment suggestion through an analysis of textual data, according to various embodiments. As illustrated in FIG. 8, the method S100 is a method of providing mental health care. The method S100 includes, at step S110, obtaining textual data from a patient; at step S120, analyzing the textual data via an algorithm to generate predictive contents, wherein the predictive contents comprise a prediction of a diagnostic condition, a prediction of a symptomatic score, and a prediction of a compliance level of the patient; at step S130, predicting a mental status of the patient in accordance with the predictive contents; and at step S140, providing a modality of mental health treatment based on the predicted mental status of the patient.

In various embodiments of the method S100, providing the modality of mental health treatment can include taking into consideration a score related to a set of quantitative measurements designed for mental health therapy. In various embodiments, the set of quantitative measurements comprises Patient Health Questionnaire-9 (PHQ-9) questions designed for depression, General Anxiety Disorder-7 (GAD-7) questions for generalized anxiety, or Quality of Life Enjoyment and Satisfaction Questionnaire-18 (Q-LES) questions for quality of life. In various embodiments, the modality of the mental health treatment is determined based on the predicted mental status of the patient and the score from one of PHQ-7, GAD-7, or Q-LES.

In various embodiments of the method S100, the prediction of the diagnostic condition includes a prediction of one or more of a physical symptom, an emotional symptom or a physical condition. In various embodiments, the physical symptom includes panic attack or anxiety. In various embodiments, the emotional symptom includes a feeling of isolation or a feeling of social phobia. In various embodiments, the physical condition includes a difficulty of sleeping or insomnia.

In various embodiments, the prediction of the symptomatic score comprises a percentage prediction for a predicted diagnostic condition. In various embodiments, the compliance level of the patient is a propensity or a likelihood that the patient stays within a therapy program.

In various embodiments, the algorithm is a natural language processing algorithm trained to evaluate a relationship of a textual statement in accordance with a five-part model of cognitive behavioral therapy that recognizes situation (positive or negative), behavior, thought (positive or negative), emotion (positive or negative), and physical reaction.

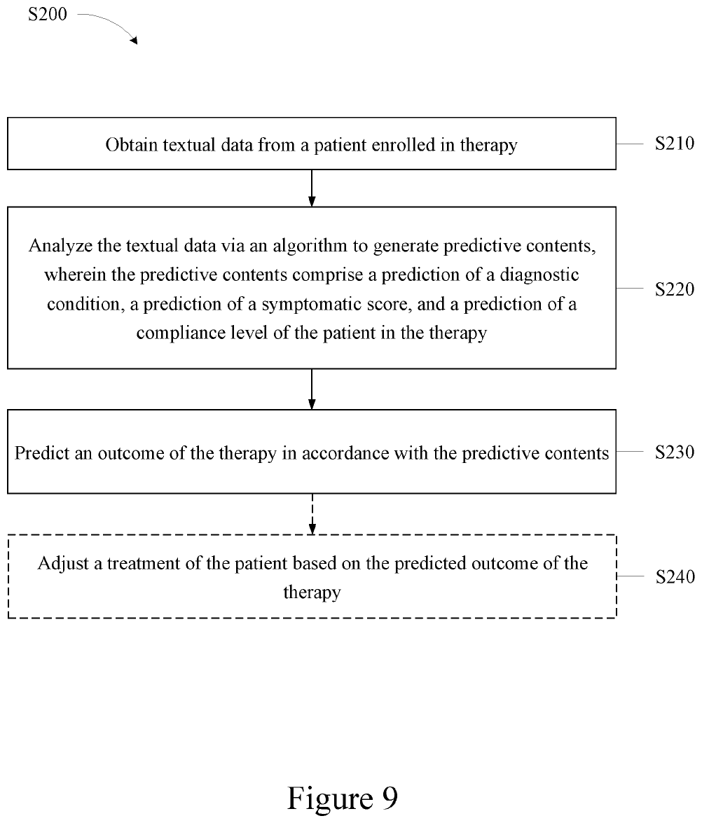

FIG. 9 illustrates a method S200 for predicting a therapy outcome through an analysis of textual data, according to various embodiments. As illustrated in FIG. 9, the method S200 is a method of providing mental health therapy. The method S200 includes, at step S210, obtaining textual data from a patient enrolled in therapy; at step S220, analyzing the textual data via an algorithm to generate predictive contents, wherein the predictive contents comprise a prediction of a diagnostic condition, a prediction of a symptomatic score, and a prediction of a compliance level of the patient in the therapy; and at step S230, predicting an outcome of the therapy in accordance with the predictive contents.

In various embodiments of the method S200, predicting the outcome of the therapy comprises taking into consideration a score related to a set of quantitative measurements designed for mental health therapy. In various embodiments, the set of quantitative measurements comprises Patient Health Questionnaire-9 (PHQ-9) questions designed for depression, General Anxiety Disorder-7 (GAD-7) questions for generalized anxiety, or Quality of Life Enjoyment and Satisfaction Questionnaire-18 (Q-LES) questions for quality of life. In various embodiments, the outcome of the therapy is predicted based on the symptomatic score and the score from one of PHQ-7, GAD-7, or Q-LES.

In various embodiments of the method S200, the prediction of the diagnostic condition comprises a prediction of one or more of a physical symptom, an emotional symptom or a physical condition. In various embodiments, the physical symptom includes panic attack or anxiety. In various embodiments, the emotional symptom includes a feeling of isolation or a feeling of social phobia. In various embodiments, the physical condition includes a difficulty of sleeping or insomnia.

In various embodiments, the prediction of the symptomatic score comprises a percentage prediction for a predicted diagnostic condition. In various embodiments, the compliance level of the patient is a propensity or a likelihood that the patient stays in therapy.

In various embodiments, the algorithm comprises a natural language processing algorithm trained to evaluate a relationship of a textual statement in accordance with a five-part model of cognitive behavioral therapy that recognizes situation (positive or negative), behavior, thought (positive or negative), emotion (positive or negative), and physical reaction.

In various embodiments, the predicted outcome of the therapy includes whether the patient completes the therapy or whether a mental status of the patient would significantly change following therapy.

In various embodiments, the method S200 may optionally include, at step S240, adjusting a treatment of the patient based on the predicted outcome of the therapy. In various embodiments, adjusting the treatment of the patient includes escalating an intervention level or adding medication to the therapy.

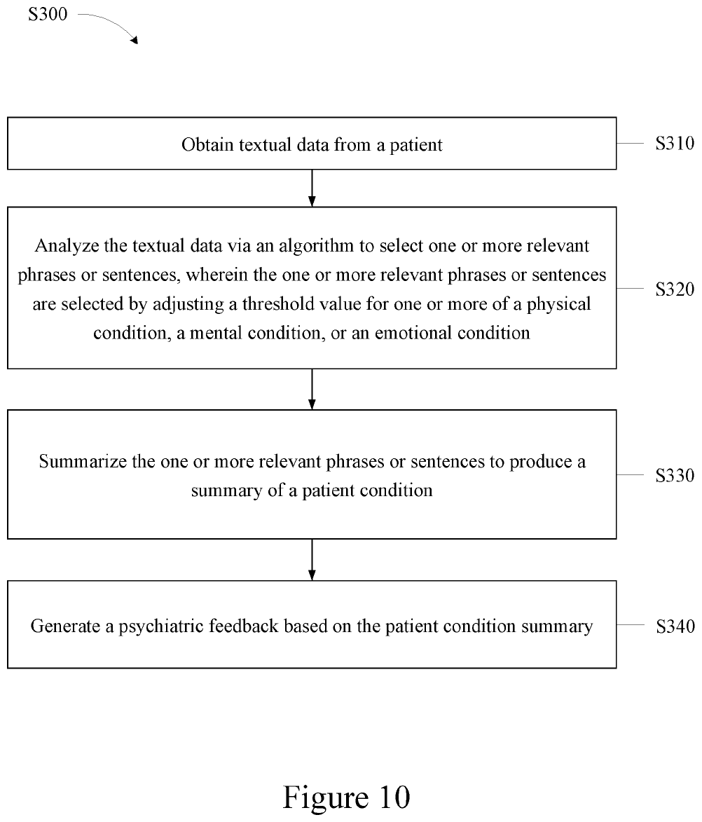

FIG. 10 illustrates a method S300 for predicting psychiatric feedback through an analysis of textual data, according to various embodiments. As illustrated in FIG. 10, the method S300 includes, at step S310, obtaining textual data from a patient; at step S320, analyzing the textual data via an algorithm to select one or more relevant phrases or sentences, wherein the one or more relevant phrases or sentences are selected by adjusting a threshold value for one or more of a physical condition, a mental condition, or an emotional condition; at step S330, summarizing the one or more relevant phrases or sentences to produce a summary of a patient condition; and at step S340, generating a psychiatric feedback based on the patient condition summary. In various embodiments, the generated psychiatric feedback is used in providing a psychiatric therapy to the patient. In various embodiments, the method S300 may optionally include, at step S350, analyzing the textual data via the algorithm to generate predictive contents, wherein the predictive contents comprise a prediction of a diagnostic condition and a prediction of a symptomatic score, wherein the predictive contents are used in providing psychiatric therapy to the patient.

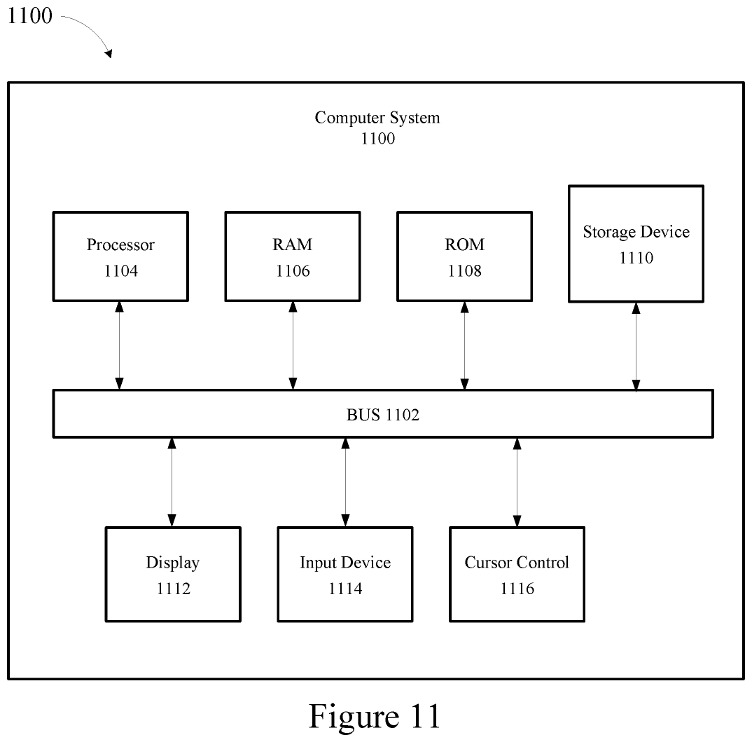

FIG. 11 is a block diagram illustrating a computer system 1100 configured to perform a method of predicting a patient/treatment response to a therapeutic, with which embodiments of the disclosed systems and methods, or portions thereof may be implemented, in accordance with various embodiments. For example, the illustrated computer system can be a local or remote computer system operatively connected to a control system for controlling or monitoring the systems and methods of the various embodiments herein. In various embodiments of the present teachings, computer system 1100 can include a bus 1102 or other communication mechanism for communicating information and a processor 1104 coupled with bus 1102 for processing information. In various embodiments, computer system 1100 can also include a memory, which can be a random-access memory (RAM) 1106 or other dynamic storage device, coupled to bus 1102 for determining instructions to be executed by processor 1104. Memory can also be used for storing temporary variables or other intermediate information during execution of instructions to be executed by processor 1104. In various embodiments, computer system 1100 can further include a read only memory (ROM) 1108 or other static storage device coupled to bus 1102 for storing static information and instructions for processor 1104. A storage device 1110, such as a magnetic disk or optical disk, can be provided and coupled to bus 1102 for storing information and instructions.

In various embodiments, computer system 1100 can be coupled via bus 1102 to a display 1112, such as a cathode ray tube (CRT) or liquid crystal display (LCD), for displaying information to a computer user. An input device 1114, including alphanumeric and other keys, can be coupled to bus 1102 for communication of information and command selections to processor 1104. Another type of user input device is a cursor control 1116, such as a mouse, a trackball or cursor direction keys for communicating direction information and command selections to processor 1104 and for controlling cursor movement on display 1112. This input device 1114 typically has two degrees of freedom in two axes, a first axis (i.e., x) and a second axis (i.e., y), that allows the device to specify positions in a plane. However, it should be understood that input devices 1114 allowing for 3-dimensional (x, y and z) cursor movement are also contemplated herein. In accordance with various embodiments, components 1112/1114/1116, together or individually, can make up a control system that connects the remaining components of the computer system to the systems herein and methods conducted on such systems, and controls execution of the methods and operation of the associated system.

Consistent with certain implementations of the present teachings, results can be provided by computer system 1100 in response to processor 1104 executing one or more sequences of one or more instructions contained in memory 1106. Such instructions can be read into memory 1106 from another computer-readable medium or computer-readable storage medium, such as storage device 1110. Execution of the sequences of instructions contained in memory 1106 can cause processor 1104 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 1104 for execution. Such a medium can take many forms, including but not limited to, non-volatile media, volatile media, and transmission media. Examples of non-volatile media can include, but are not limited to, dynamic memory, such as memory 1106. Examples of transmission media can include, but are not limited to, coaxial cables, copper wire, and fiber optics, including the wires that comprise bus 1102.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, another memory chip or cartridge, or any other tangible medium from which a computer can read.

In addition to computer-readable medium, instructions or data can be provided as signals on transmission media included in a communications apparatus or system to provide sequences of one or more instructions to processor 1104 of computer system 1100 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein, flow charts, diagrams and accompanying disclosure can be implemented using computer system 1100 as a standalone device or on a distributed network or shared computer processing resources such as a cloud computing network. In one or more embodiments, the computer system 1100 may include a single computer (or computer system) or multiple computers in communication with each other in a distributed implementation.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 1100, whereby processor 1104 would execute the analyses and determinations provided by these engines, subject to instructions provided by any one of, or a combination of, memory components 1106/1108/1110 and user input provided via input device 1114.

While the present teachings are described in conjunction with various embodiments, it is not intended that the present teachings be limited to such various embodiments. On the contrary, the present teachings encompass various alternatives, modifications, and equivalents, as will be appreciated by those of skill in the art.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

### Recitation of Embodiments

Embodiment 1. A method of providing mental health care, comprising: obtaining textual data from a patient; analyzing the textual data via an algorithm to generate predictive contents, wherein the predictive contents comprise a prediction of a diagnostic condition, a prediction of a symptomatic score, and a prediction of a compliance level of the patient; predicting a mental status of the patient in accordance with the predictive contents; and providing a modality of mental health treatment based on the predicted mental status of the patient.

Embodiment 2. The method of Embodiment 1, wherein providing the modality of mental health treatment further comprises taking into consideration a score related to a set of quantitative measurements designed for mental health therapy.

Embodiment 3. The method of Embodiment 2, wherein the set of quantitative measurements comprises Patient Health Questionnaire-9 (PHQ-9) questions designed for depression, General Anxiety Disorder-7 (GAD-7) questions for generalized anxiety, or Quality of Life Enjoyment and Satisfaction Questionnaire-18 (Q-LES) questions for quality of life.

Embodiment 4. The method of Embodiment 2, wherein the modality of the mental health treatment is determined based on the predicted mental status of the patient and the score from one of PHQ-7, GAD-7, or Q-LES.

Embodiment 5. The method of any one of Embodiments 1-4, wherein the prediction of the diagnostic condition comprises a prediction of a physical symptom, an emotional symptom or a physical condition.

Embodiment 6. The method of Embodiment 5, wherein the physical symptom includes panic attack or anxiety.

Embodiment 7. The method of any one of Embodiments 1-6, wherein the emotional symptom includes a feeling of isolation or a feeling of social phobia.

Embodiment 8. The method of any one of Embodiments 1-7, wherein the physical condition includes a difficulty of sleeping or insomnia.

Embodiment 9. The method of any one of Embodiments 1-8, wherein the prediction of the symptomatic score comprises a percentage prediction for a predicted diagnostic condition.

Embodiment 10. The method of any one of Embodiments 1-9, wherein the compliance level of the patient is a propensity or a likelihood that the patient stays within a therapy program.

Embodiment 11. The method of any one of Embodiments 1-10, wherein the algorithm comprises a natural language processing algorithm trained to evaluate a relationship of a textual statement in accordance with a five-part model of cognitive behavioral therapy that recognizes situation (positive or negative), behavior, thought (positive or negative), emotion (positive or negative), and physical reaction.

Embodiment 12. A method of providing mental health therapy, comprising: obtaining textual data from a patient enrolled in therapy; analyzing the textual data via an algorithm to generate predictive contents, wherein the predictive contents comprise a prediction of a diagnostic condition, a prediction of a symptomatic score, and a prediction of a compliance level of the patient in the therapy; and predicting an outcome of the therapy in accordance with the predictive contents.

Embodiment 13. The method of Embodiment 12, wherein predicting the outcome of the therapy comprises taking into consideration a score related to a set of quantitative measurements designed for mental health therapy.

Embodiment 14. The method of Embodiment 13, wherein the set of quantitative measurements comprises Patient Health Questionnaire-9 (PHQ-9) questions designed for depression, General Anxiety Disorder-7 (GAD-7) questions for generalized anxiety, or Quality of Life Enjoyment and Satisfaction Questionnaire-18 (Q-LES) questions for quality of life.

Embodiment 15. The method of Embodiment 14, wherein the outcome of the therapy is predicted based on the symptomatic score and the score from one of PHQ-7, GAD-7, or Q-LES.

Embodiment 16. The method of any one of Embodiments 12-15, wherein the prediction of the diagnostic condition comprises a prediction of a physical symptom, an emotional symptom or a physical condition.

Embodiment 17. The method of any one of Embodiments 12-16, wherein the physical symptom includes panic attack or anxiety.

Embodiment 18. The method of any one of Embodiments 12-17, wherein the emotional symptom includes a feeling of isolation or a feeling of social phobia.

Embodiment 19. The method of any one of Embodiments 12-18, wherein the physical condition includes a difficulty of sleeping or insomnia.

Embodiment 20. The method of any one of Embodiments 12-19, wherein the prediction of the symptomatic score comprises a percentage prediction for a predicted diagnostic condition.

Embodiment 21. The method of any one of Embodiments 12-20, wherein the compliance level of the patient is a propensity or a likelihood that the patient stays in therapy.

Embodiment 22. The method of any one of Embodiments 12-21, wherein the algorithm comprises a natural language processing algorithm trained to evaluate a relationship of a textual statement in accordance with a five-part model of cognitive behavioral therapy that recognizes situation (positive or negative), behavior, thought (positive or negative), emotion (positive or negative), and physical reaction.

Embodiment 23. The method of any one of Embodiments 12-22, wherein the predicted outcome of the therapy includes whether the patient completes the therapy or whether a mental status of the patient would significantly change following therapy.

Embodiment 24. The method of any one of Embodiments 12-23, further comprising: adjusting a treatment of the patient based on the predicted outcome of the therapy.

Embodiment 25. The method of Embodiment 24, wherein adjusting the treatment of the patient includes escalating an intervention level or adding medication to the therapy.

Embodiment 26. A method of providing psychiatric feedback, comprising: obtaining textual data from a patient; analyzing the textual data via an algorithm to select one or more relevant phrases or sentences, wherein the one or more relevant phrases or sentences are selected by adjusting a threshold value for one or more of a physical condition, a mental condition, or an emotional condition; summarizing the one or more relevant phrases or sentences to produce a summary of a patient condition; and generating a psychiatric feedback based on the patient condition summary.

Embodiment 27. The method of Embodiment 26, wherein the generated psychiatric feedback is used in providing a psychiatric therapy to the patient.

Embodiment 28. The method of Embodiments 26 or 27, further comprising: analyzing the textual data via the algorithm to generate predictive contents, wherein the predictive contents comprise a prediction of a diagnostic condition and a prediction of a symptomatic score, wherein the predictive contents are used in providing psychiatric therapy to the patient.

Embodiment 29. A system for providing mental health care, the system comprising a computing device configured to perform operations for providing the mental health care, wherein the operations comprise: obtaining textual data from a patient; analyzing the textual data via an algorithm to generate predictive contents, wherein the predictive contents comprise a prediction of a diagnostic condition, a prediction of a symptomatic score, and a prediction of a compliance level of the patient; predicting a mental status of the patient in accordance with the predictive contents; and providing a modality of mental health treatment based on the predicted mental status of the patient.

Embodiment 30. The system of Embodiment 29, wherein providing the modality of mental health treatment further comprises taking into consideration a score related to a set of quantitative measurements designed for mental health therapy.

Embodiment 31. The system of Embodiment 30, wherein the set of quantitative measurements comprises Patient Health Questionnaire-9 (PHQ-9) questions designed for depression, General Anxiety Disorder-7 (GAD-7) questions for generalized anxiety, or Quality of Life Enjoyment and Satisfaction Questionnaire-18 (Q-LES) questions for quality of life.

Embodiment 32. The system of Embodiment 30, wherein the modality of the mental health treatment is determined based on the predicted mental status of the patient and the score from one of PHQ-7, GAD-7, or Q-LES.

Embodiment 33. The system of any one of Embodiments 29-32, wherein the prediction of the diagnostic condition comprises a prediction of a physical symptom, an emotional symptom or a physical condition.

Embodiment 34. The system of Embodiment 33, wherein the physical symptom includes panic attack or anxiety.

Embodiment 35. The system of any one of Embodiments 29-34, wherein the emotional symptom includes a feeling of isolation or a feeling of social phobia.

Embodiment 36. The system of any one of Embodiments 29-35, wherein the physical condition includes a difficulty of sleeping or insomnia.

Embodiment 37. The system of any one of Embodiments 29-36, wherein the prediction of the symptomatic score comprises a percentage prediction for a predicted diagnostic condition.

Embodiment 38. The system of any one of Embodiments 29-37, wherein the compliance level of the patient is a propensity or a likelihood that the patient stays within a therapy program.

Embodiment 39. The system of any one of Embodiments 29-38, wherein the algorithm comprises a natural language processing algorithm trained to evaluate a relationship of a textual statement in accordance with a five-part model of cognitive behavioral therapy that recognizes situation (positive or negative), behavior, thought (positive or negative), emotion (positive or negative), and physical reaction.

Embodiment 40. A system for providing mental health therapy, the system comprising a computing device configured to perform operations for providing the mental health therapy, wherein the operations comprise: obtaining textual data from a patient enrolled in therapy; analyzing the textual data via an algorithm to generate predictive contents, wherein the predictive contents comprise a prediction of a diagnostic condition, a prediction of a symptomatic score, and a prediction of a compliance level of the patient in the therapy; and predicting an outcome of the therapy in accordance with the predictive contents.

Embodiment 41. The system of Embodiment 40, wherein predicting the outcome of the therapy comprises taking into consideration a score related to a set of quantitative measurements designed for mental health therapy.

Embodiment 42. The system of Embodiment 41, wherein the set of quantitative measurements comprises Patient Health Questionnaire-9 (PHQ-9) questions designed for depression, General Anxiety Disorder-7 (GAD-7) questions for generalized anxiety, or Quality of Life Enjoyment and Satisfaction Questionnaire-18 (Q-LES) questions for quality of life.

Embodiment 43. The system of Embodiment 42, wherein the outcome of the therapy is predicted based on the symptomatic score and the score from one of PHQ-7, GAD-7, or Q-LES.

Embodiment 44. The system of any one of Embodiments 40-43, wherein the prediction of the diagnostic condition comprises a prediction of a physical symptom, an emotional symptom or a physical condition.

Embodiment 45. The system of any one of Embodiments 40-44, wherein the physical symptom includes panic attack or anxiety.

Embodiment 46. The system of any one of Embodiments 40-45, wherein the emotional symptom includes a feeling of isolation or a feeling of social phobia.

Embodiment 47. The system of any one of Embodiments 40-46, wherein the physical condition includes a difficulty of sleeping or insomnia.

Embodiment 48. The system of any one of Embodiments 40-47, wherein the prediction of the symptomatic score comprises a percentage prediction for a predicted diagnostic condition.

Embodiment 49. The system of any one of Embodiments 40-48, wherein the compliance level of the patient is a propensity or a likelihood that the patient stays in therapy.

Embodiment 50. The system of any one of Embodiments 40-49, wherein the algorithm comprises a natural language processing algorithm trained to evaluate a relationship of a textual statement in accordance with a five-part model of cognitive behavioral therapy that recognizes situation (positive or negative), behavior, thought (positive or negative), emotion (positive or negative), and physical reaction.

Embodiment 51. The system of any one of Embodiments 40-50, wherein the predicted outcome of the therapy includes whether the patient completes the therapy or whether a mental status of the patient would significantly change following therapy.

Embodiment 52. The system of any one of Embodiments 40-51, further comprising: adjusting a treatment of the patient based on the predicted outcome of the therapy.

Embodiment 53. The system of Embodiment 52, wherein adjusting the treatment of the patient includes escalating an intervention level or adding medication to the therapy.

Embodiment 54. A system for providing psychiatric feedback, the system comprising a computing device configured to perform operations for providing the psychiatric feedback, wherein the operations comprise: obtaining textual data from a patient; analyzing the textual data via an algorithm to select one or more relevant phrases or sentences, wherein the one or more relevant phrases or sentences are selected by adjusting a threshold value for one or more of a physical condition, a mental condition, or an emotional condition; summarizing the one or more relevant phrases or sentences to produce a summary of a patient condition; and generating a psychiatric feedback based on the patient condition summary.

Embodiment 55. The system of Embodiment 54, wherein the generated psychiatric feedback is used in providing a psychiatric therapy to the patient.

Embodiment 56. The system of Embodiments 54 or 55, further comprising: analyzing the textual data via the algorithm to generate predictive contents, wherein the predictive contents comprise a prediction of a diagnostic condition and a prediction of a symptomatic score, wherein the predictive contents are used in providing psychiatric therapy to the patient.

What is claimed is:

1. A method of providing mental health care, comprising:
obtaining textual data from a patient;
analyzing the textual data via an algorithm to generate predictive contents, wherein the predictive contents comprise a prediction of a diagnostic condition, a prediction of a symptomatic score, and a prediction of a compliance level of the patient;
predicting a mental status of the patient in accordance with the predictive contents; and
providing a modality of mental health treatment based on the predicted mental status of the patient.

2. The method of claim 1, wherein providing the modality of mental health treatment further comprises taking into consideration a score related to a set of quantitative measurements designed for mental health therapy.

3. The method of claim 2, wherein the set of quantitative measurements comprises Patient Health Questionnaire-9 (PHQ-9) questions designed for depression, General Anxiety Disorder-7 (GAD-7) questions for generalized anxiety, or Quality of Life Enjoyment and Satisfaction Questionnaire-18 (Q-LES) questions for quality of life.

4. The method of claim 3, wherein the modality of the mental health treatment is determined based on the predicted mental status of the patient and the score from one of PHQ-7, GAD-7, or Q-LES.

5. The method of claim 1, wherein the prediction of the diagnostic condition comprises a prediction of a physical symptom, an emotional symptom or a physical condition.

6. The method of claim 5, wherein the physical symptom includes panic attack or anxiety, the emotional symptom includes a feeling of isolation or a feeling of social phobia, or the physical condition includes a difficulty of sleeping or insomnia.

7. The method of claim 1, wherein the prediction of the symptomatic score comprises a percentage prediction for a predicted diagnostic condition.

8. The method of claim 1, wherein the compliance level of the patient is a propensity or a likelihood that the patient stays within a therapy program.

9. The method of claim 1, wherein the algorithm comprises a natural language processing algorithm trained to evaluate a relationship of a textual statement in accordance with a five-part model of cognitive behavioral therapy that recognizes situation (positive or negative), behavior, thought (positive or negative), emotion (positive or negative), and physical reaction.

10. A method of providing mental health therapy, comprising:
obtaining textual data from a patient enrolled in therapy;
analyzing the textual data via an algorithm to generate predictive contents, wherein the predictive contents comprise a prediction of a diagnostic condition, a prediction of a symptomatic score, and a prediction of a compliance level of the patient in the therapy; and
predicting an outcome of the therapy in accordance with the predictive contents.

11. The method of claim 10, wherein predicting the outcome of the therapy comprises taking into consideration a score related to a set of quantitative measurements designed for mental health therapy, wherein the set of quantitative measurements comprises Patient Health Questionnaire-9 (PHQ-9) questions designed for depression, General Anxiety Disorder-7 (GAD-7) questions for generalized anxiety, or Quality of Life Enjoyment and Satisfaction Questionnaire-18 (Q-LES) questions for quality of life.

12. The method of claim 11, wherein the outcome of the therapy is predicted based on the symptomatic score and the score from one of PHQ-7, GAD-7, or Q-LES.

13. The method of claim 10, wherein the prediction of the diagnostic condition comprises a prediction of a physical symptom, an emotional symptom or a physical condition.

14. The method of claim 13, wherein the physical symptom includes panic attack or anxiety, the emotional symptom includes a feeling of isolation or a feeling of social phobia, or the physical condition includes a difficulty of sleeping or insomnia.

15. The method of claim 10, wherein the prediction of the symptomatic score comprises a percentage prediction for a predicted diagnostic condition.

16. The method of claim 10, wherein the compliance level of the patient is a propensity or a likelihood that the patient stays in therapy.

17. The method of claim 10, wherein the algorithm comprises a natural language processing algorithm trained to evaluate a relationship of a textual statement in accordance with a five-part model of cognitive behavioral therapy that recognizes situation (positive or negative), behavior, thought (positive or negative), emotion (positive or negative), and physical reaction.

18. The method of claim 10, wherein the predicted outcome of the therapy includes whether the patient completes the therapy or whether a mental status of the patient would significantly change following therapy.

19. The method of claim 10, further comprising:
adjusting a treatment of the patient based on the predicted outcome of the therapy, wherein adjusting the treatment of the patient includes escalating an intervention level or adding medication to the therapy.

20. A method of providing psychiatric feedback, comprising:
obtaining textual data from a patient;
analyzing the textual data via an algorithm to select one or more relevant phrases or sentences, wherein the one or more relevant phrases or sentences are selected by adjusting a threshold value for one or more of a physical condition, a mental condition, or an emotional condition;
summarizing the one or more relevant phrases or sentences to produce a summary of a patient condition; and
generating a psychiatric feedback based on the patient condition summary.

21. The method of claim 20, wherein the generated psychiatric feedback is used in providing a psychiatric therapy to the patient.

22. The method of claim 20, further comprising:
analyzing the textual data via the algorithm to generate predictive contents, wherein the predictive contents comprise a prediction of a diagnostic condition and a prediction of a symptomatic score, wherein the predictive contents are used in providing psychiatric therapy to the patient.
